(19) United States
(12) Patent Application Publication
(10) Pub. No.: US 2019/0076678 A1
(43) Pub. Date: Mar. 14, 2019

(54) DERMATOLOGICAL COMPOSITION AUGMENTING PARACRINE SIGNALLING

(71) Applicant: Serucell Corporation, Palo Alto, CA (US)

(72) Inventor: Walter De Paula Neto, Lavalette, WV (US)

(21) Appl. No.: 16/127,906

(22) Filed: Sep. 11, 2018

Related U.S. Application Data
(60) Provisional application No. 62/556,770, filed on Sep. 11, 2017.

(51) Int. Cl. A61Q 19/08; A61K 9/00; A61K 38/18; A61K 38/39; A61K 38/48

(52) U.S. Cl. CPC A61Q 19/08; A61K 9/0014; A61K 38/4886; A61K 38/1866; A61K 38/39; A61K 38/1841

(57) ABSTRACT

A composition facilitating healthy skin by augmenting natural paracrine to improve and/or maintain replenishment by utilizing a therapeutic effective amount of at a least one an angio-modifying formulation, a morphogenesis formulation, an extracellular matrix formulation, an extracellular matrix modification formulation, an immune promoting formulation and a cytoskeleton formulation.

### CROSS REFERENCE TO RELATED APPLICATIONS

This application claims priority to U.S. Provisional Patent. Application No. 62/556,770, filed Sep. 11, 2017, the disclosure of which is hereby incorporated by reference in its entirety.

### FIELD

A dermatological composition augmenting natural paracrine signaling to improve and/or maintain replenishment of the skin.

### BACKGROUND

Skin is an essential multilayer organ. Providing a barrier against pathogens and toxins, as well as synthesizing nutrients such as vitamin D, skin is essential for maintaining an individual's physical health. The integrity of skin is also essential for maintaining one's psychological health. Skin is the most prominent part of an individual's body. Blemishes, scars, wrinkles and perceived imperfections can diminish an individual's self-confidence. Maintaining physical and psychological health, therefore, requires maintaining healthy skin.

Healthy skin comprises layers of different cells supported by a scaffolding of proteins called the extracellular matrix. The extracellular matrix supporting the skin comprises various proteins such as collagen, fibronectin and laminin. These and other structural proteins intertwine and communicate to form the structural and dynamic three-dimensional scaffolding providing skin with its strength and resilience. Cells forming the various layers of skin adhere to the extracellular matrix, and rely on both structural and biologically active signal relaying molecules within the matrix to maintain proper function. During wound healing, cells also use the extracellular matrix as a bridge to migrate into and close wounds. Providing support and pathways for healing, the extracellular matrix is an important structural and biologically active component of healthy skin. As an individual ages, however, the extracellular matrix changes and becomes weakened, leading to the appearance of wrinkles, blemishes and decreased healing.

The extracellular matrix is manufactured and maintained primarily by fibroblasts beneath the skin's surface. Manufacturing and maintaining the biological activity of the extracellular matrix, healthy fibroblasts are essential for healthy skin. Above the fibroblasts, on top of the extracellular matrix, are keratinocytes forming the epidermis. Forming the epidermis, the outer layer of skin, healthy keratinocytes are also essential for the appearance and integrity of skin. Accordingly, maintaining healthy skin from its base to its surface requires promoting the health of different cells throughout the layers of skins.

As to maintain the health of different cells, and the extracellular matrix, skin naturally replenishes itself. During replenishment new cells are created to replace old and lost cells. The skin is also replenished by the delivery of nutrients and moisture from capillaries. Additionally, the extracellular matrix is replenished by the incorporation of new structural proteins as to preserve the integrity and strength of skin. Providing moisture, nutrients and new cells, while also maintaining the extracellular matrix, replenishment of the skin is necessary for maintaining skin. When the skin is not properly replenished several skin conditions such as wrinkles, eczema, acne, impetigo, psoriasis, rosacea, rashes and/or other skin conditions may develop. As these skin conditions affect that appearance of the skin, the can be particularly detrimental to an individual's self-confidence and psychological health.

### SUMMARY

Replenishment of the skin is controlled by paracrine signaling within the skin. A composition augmenting natural paracrine signaling, accordingly, may facilitate healthy skin by improving and/or maintaining replenishment of the skin. Replenishing the skin may require angio-modifications, morphogenesis of cells, changes to the formulation of the extracellular matrix, modifications of the extracellular matrix, activation of the immune system and modifications to cytoskeletons. Each of these different types of changes may be induced by paracrine signaling. Maintaining and/or improving replenishment of skin, therefore, may be accomplished with a dermatological composition augmenting paracrine signaling comprising a therapeutic effective amount of at a least one an angio-modifying formulation, a morphogenesis formulation, an extracellular matrix formulation, an extracellular matrix modification formulation, an immune promoting formulation and a cytoskeleton formulation. Furthermore, applying a dermatological composition comprising a therapeutic effective amount of at a least one an angio-modifying formulation, a morphogenesis formulation, an extracellular matrix formulation, an extracellular matrix modification formulation, an immune promoting formulation and a cytoskeleton formulation may treat at least one of wrinkles, eczema, acne, impetigo, psoriasis, rashes, rosacea, hives and other skin conditions.

The angio-modifying formulation, morphogenesis formulation, extracellular matrix formulation, extracellular matrix modification formulation, immune promoting formulation and/or cytoskeleton formulation may comprise proteins having catalytic, structural, regulatory and/or messaging functions. Proteins with a catalytic function may catalyze reactions within the skin. For instance, catalytic proteins may catalyze the degradation of the extracellular matrix. Structural proteins within one or more of the formulations may become incorporated into the structure and arrangement of the skin. For instance, structural proteins may become part of the cytoskeleton, extracellular matrix and/or junctions between cells. Structural proteins may also anchor cells to the extracellular matrix. Regulatory proteins within the formulations may control the action of proteins within the skin. For instance, a regulatory protein may activate or inhibit a catalytic protein. Proteins providing messaging functions may be responsible for activating processes within the skin. A messaging protein within the formulations, for instance, may bind to receptors within the skin to elicit and/or inhibit various cellular process. For instance, a messaging protein may act attract immune cells to the skin.

As the varied actions of proteins that may be present within the formulations of the composition demonstrate, augmenting paracrine signaling may be accomplished by doing more than just amplifying the paracrine signaling naturally present. A composition amplifying natural paracrine would comprise at least one formulation having at least one messenger protein of natural paracrine signal. Generally, cells respond proportionally to the amount of the messenger proteins received. Accordingly, if more messenger proteins were present, then the cell would produce a bigger response. Compositions comprising a messenger protein of natural paracrine signaling would, consequently, augment paracrine signaling by increasing one aspect of natural replenishment. In addition, or the alternative, to amplifying one aspect of natural paracrine signaling, the composition may augment paracrine signaling by providing catalytic activities, regulation, messages and/or structural changes not induced by normal paracrine signaling. The augmentation of paracrine signaling provided by the composition may improve the efficiency of replenishment lost with aging. It is also possible to augment paracrine signaling by providing proteins naturally produced by paracrine signaling. Compositions may augment paracrine through any combination of these mechanisms.

During paracrine signaling, molecules communicating massages about the environment and/or health the of skin move between cells and layers. When the signaling molecules reach their target cells and/or layers, an appropriate response is induced in the receiving cell. The molecules may travel between cells and/or layers by diffusion, i.e. movement through a fluid. Thus, to have proper paracrine signaling the skin needs to remain hydrated, as to provide a fluid through which the signaling molecules can move. If the skin were to become dry, a symptom of aging, wrinkles, rashes and eczema, the loss of fluid would limit the diffusion of signaling molecules. The reduced diffusion of signaling molecules in dry skin may delay, diminish and/or prevent paracrine signaling. The resulting impaired signaling may diminish replenishment, further deteriorating skin health. A capillary bed beneath the skin supplies the skin with nutrients and moisture, keeping the skin hydrated and nourished. Modifying the capillary bed to increase its ability to supply the skin with moisture and nutrients may, therefore, augment paracrine signaling to improve the efficiency of replenishment lost with aging and/or due to skin conditions. Accordingly, augmenting paracrine signaling may be accomplished with a dermatological composition comprising a therapeutic effective amount of angio-modifying formulation comprising agrin and calpastatin. Furthermore, applying a dermatological composition comprising a therapeutic effective amount of angio-modifying formulation comprising agrin and calpastatin to the skin may treat at least one of wrinkles, rashes, eczema and other skin conditions.

The ability of calpastatin in combination with agrin to augment paracrine signaling to improve the efficiency of replenishment by modifying the capillary bed is unexpected. Calpastatin has been shown to impair angiogenesis. Impairing angiogenesis, calpastatin would be expect to prevent modification of the capillary bed, resulting in impaired skin health. Dermatological compositions comprising calpastatin and agrin, however, improve replenishment of the skin.

In addition to agrin and calpastatin, the angio-modifying formulation may comprise at least one of protein Jagged-1, Isoform 2 of growth arrest-specific protein 6, vascular endothelial growth factor C, 72 kDa type IV collagenase, desmoglein-2.

In addition to facilitating the movement of signaling molecules, paracrine signaling directing replenishment of the skin must induce cell mobility and differentiating, so that new cells can replace lost cells. As the skin replenishes itself, newly formed cells move upwards to replace lost cells. During upward movement from lower layers, the newly formed cells differentiate, i.e., undergo morphological changes, to develop the morphology characteristic of the outer layer of the skin. Accordingly, providing the skin with morphological proteins promoting differentiation and cell mobility may augment paracrine signaling to improve the efficiency of replenishment lost with aging and/or due to various skin conditions. Fibronectin, a protein of the extracellular matrix, facilitates cell mobility by providing footholds for cells moving upwards from lower layers. Tenascin, however, inhibits cellular adhesion to fibronectin. Accordingly, it would be expected that tenascin would inhibit cell mobility by causing the cells replenishing the skin to fall off fibronectin. Dermatological compositions comprising tenascin isoform 4 and Fibronectin isoform 3, however, improve replenishment of the skin. Augmenting paracrine signaling, therefore, may be accomplished with a dermatological composition comprising a therapeutic effective amount of a morphogenesis formulation comprising tenascin isoform 4 and fibronectin isoform 3. Furthermore, applying a dermatological composition comprising a therapeutic effective amount of a morphogenesis formulation comprising tenascin isoform 4 and fibronectin isoform 3 to the skin may treat at least one of wrinkles, eczema, acne, psoriasis and other skin conditions.

In addition to tenascin isoform 4 and fibronectin isoform 3, the morphogenesis formulation may comprise at least one of transforming growth factor beta induced protein ig-h3, plasminogen activator inhibitor 1, amyloid beta A4 protein, glucose-6-phosphate isomerase, long isoform of serine protease inhibitor Kazal-type 5, cadherin-3, pappalysin-1, insulin-like growth factor-binding protein 7, kallikrein-10, protocadherin fat 1, syntenin-1, proliferation-associated protein 2G4 protein CYR61, keratinocyte proline-rich protein, brain-specific serein protease 4, cadherin 13 isoform 4, integrin alpha-2, integrin beta-1, and neural cell adhesion molecule L1 isoform 2.

In addition to facilitating morphogenesis of cells and/or movement of signaling molecules, paracrine signaling directing replenishment of the skin must maintain the integrity of the extracellular matrix, so that new cells are not lost to rapidly. If this scaffolding holding the skin together were to weaken, replenished cells would quickly shed, loose it elasticity and wrinkle. Decreases in the integrity of skin is a symptom of aging. One of the proteins holding cells to the extracellular matrix is laminin. Augmenting paracrine signaling to maintain the efficiency of replenishment lost with aging and/or due to skin conditions, accordingly, may also be accomplished by maintaining the integrity of the extracellular matrix. As such, dermatological compositions having a therapeutic effective an extracellular matrix formulation comprising at least one laminin alpha 3, laminin beta 3, laminin beta 2 and laminin 332 may augment paracrine signaling to improve replenishment of the skin. Furthermore, applying a dermatological composition comprising a therapeutic effective an extracellular matrix formulation comprising at least one laminin alpha 3, laminin beta 3, laminin beta 2 and laminin 332 to the skin may treat at least one of wrinkles, eczema, impetigo, psoriasis and other skin conditions.

In addition to the afore mentioned laminins, the extracellular matrix formulation may comprise at least one of collagen alpha-1(I), collagen alpha-1(III), collagen alpha-1(VI), collagen alpha-2(I), collagen alpha-2(VI), laminin subunit alpha 4, laminin subunit beta 1, laminin subunit gamma 1, and laminin 411.

While the integrity of the extracellular matrix is important, so too is the integrity of newly formed cells. A key component of cellular strength, is the cytoskeleton. The cytoskeleton is a series of interconnected proteins assisting the cell in maintaining its integrity and shape. Additionally, proteins within the cytoskeleton may facilitate cell mobility. Accordingly, augmenting paracrine signaling may be accomplished with a dermatological composition comprising a therapeutic effective amount of a cytoskeleton formulation. Furthermore, applying a dermatological composition comprising a therapeutic effective amount of a cytoskeleton formulation to the skin may treat at least one of wrinkles, eczema, acne, impetigo, psoriasis, rashes, rosacea, hives and other skin conditions. The cytoskeleton formulation may comprise at least one of translationally-controlled tumor protein, filamin A, alpha-actin-1, microtubule associated protein 4, moesin, vinculin in a peptide, involucrin, gelsolin isoform 2, PDZ and LIM domain protein 1, caldesmon isoform 5, LIM domain and actin-binding protein 1, myosin regulatory light chain 12B, small proline rich protein 3, smooth muscle isoform of myosin light polypeptide 6, jupiter microtubule associated homolog 1, small proline rich protein 2A, myotrophin.

Although preserving and/or increasing the integrity of the skin and/or skin cells may augment paracrine signaling to maintain the efficiency of replenishment lost with aging and/or due to skin conditions, shedding of dead cells, removal of toxins and removal of microorganisms is necessary to maintain the health of the skin. Generally, such house-keeping activities result from an inflammation response induced by the complement system of the immune system. Inducing inflammation and/or other immune responses seems counterintuitive. After all, inflammation is generally characterized by redness and burning, itching and/or painful sensations. Given these negative effects of inflammation and other immune responses, one would suspect that including within the composition an immune promoting formulation would lead to a decrease skin health and/or discomfort. Despite these negative expectations, dermatological compositions comprising serapin B7, complement component C1s and complement component C3 improve replenishment of the skin. Perhaps, the improved skin replenishment results from an increased removal of old skin cells. Augmenting paracrine signaling, therefore, may be accomplished with a dermatological composition comprising a therapeutic effective amount of an immune promoting formulation comprising serapin B7, complement component C1s and complement component C3 improve replenishment of the skin. Furthermore, applying a dermatological composition comprising a therapeutic effective amount of an immune promoting formulation comprising serapin B7, complement component C1s and complement component C3 to the skin may treat at least one of eczema, acne, impetigo, psoriasis, rashes, rosacea, hives and other skin conditions.

In addition to serapin B7, complement component C1s and complement component C3, the immune promoting formulation may comprise at least one of neutrophil chemotactic agent, such as, but not limited to, interleukin-6, growth regulated alpha protein, protein S100-A8, interleukin-8, and/or C-X-C motif chemokine 5, and/or at least one of elafin, matrix metalloproteinase-9, stromelysin-2, HLA class I histocompatibility antigen, Cw-6 alpha chain, quinone oxidoreductase PIG3, superoxide dismutase, metallothionein-2, alpha-1 antichymotrypsin, interleukin-1 receptor-like 1.

Just providing the skin with extracellular matrix proteins, immune promoting proteins, angio-modifying proteins and/or cytoskeleton proteins may not be sufficient to preserve skin integrity by augmenting paracrine signaling to maintain the efficiency of replenishment lost with aging and/or due to skin conditions. The extracellular matrix proteins provided by the composition and/or synthesized by the skin may not be readily incorporated into the existing extracellular matrix. It is also possible the strengthened and/or migrating cells may need assistance traversing through the extracellular matrix. Likewise, neutrophils and/or other immune cells may need help infiltrating the skin. Additionally, the extracellular matrix may inhibit modification of the capillary bed and/or delivery of nutrients and/or moisture to the skin. Such barriers may be overcome by including within the composition extracellular matrix modifying proteins. The catalytic activity provided, directly and/or indirectly, by these proteins may facilitate incorporating new proteins of the extracellular matrix. In combination or the alternative, these proteins may facilitate passage of nutrients, moisture and/or cells through the skin. Augmenting paracrine signaling to maintain the efficiency of replenishment lost with aging, accordingly, may also be accomplished by a dermatological composition having a therapeutic effective amount of an extracellular matrix modification formulation comprising at least one interstitial collagenase and stomelysin-1. Furthermore, applying a dermatological composition having a therapeutic effective amount of an extracellular matrix modification formulation comprising at least one interstitial collagenase and stomelysin-1 to the skin may treat at least one of wrinkles, eczema, acne, impetigo, psoriasis, rashes, rosacea, hives and other skin conditions.

In addition to interstitial collagenase and stomelysin-1, the extracellular matrix modification formulation may comprise at least one of cathepsin L2, latent transforming growth factor beta-binding protein 2, aminopeptidase N, decorin, urokinase-type plasminogen activator, lumican, cystatin-M, lysyl oxidase homolog 2, cystatin-C, protein-lysine 6-oxidase, tissue factor pathway inhibitor 2, procollagen-lysine, 2-oxoglutarate 5-dioxgenase isoform 2

In addition to a therapeutic effective amount of at a least one an angio-modifying formulation, a morphogenesis formulation, an extracellular matrix formulation, an extracellular matrix modification formulation, an immune promoting formulation and a cytoskeleton formulation, the dermatological composition may further comprise a cosmetically suitable base. The cosmetic base which may be used is not particularly limited and may include hydrogels, such as polyethylene glycol, oils, such as sunflower seed oil, sweet almond oil and/or coconut oil, and/or fats, such as Shea Butter. The cosmetic base may also include alcohols, polyols, emulsifiers, such as Ceteareth-20, carbomer and/or glycerol monostearate, preservatives, and/or moisturizers, such as hyaluronic acid. Antioxidants, such as vitamin E, vitamin A and/or vitamin C may also be included in the composition. The dermatological composition may include phytochemicals, such as resveritol, quercetin and/or epigallocatechin gallate. If desired, fragrances may also be added to the dermatological composition. The dermatological composition may contain other ingredients, such as pigments, flavoring agents, preservatives and/or sweeteners. The ingredients included within the dermatological composition are not particularly limited, as long as they collectively provide a dermatologically suitable preparation that is non-toxic when topically applied.

### DETAILED DESCRIPTION

The various formulations of the dermatological composition may be obtained by combining proteins with a suitable solvent, such as water. The proteins of each formulation may be obtained from various suppliers and/or harvested from various organisms. The proteins may also be isolated from serums created by cell cultures. A serum providing at least the majority of the proteins may be produced as described below. The serum, however, may be produced in different manners, and thus should not be construed as limited to the specific examples provided. Accordingly, the serum may be obtained by a different ordering and/or sequence of the various steps and/or procedures detailed in the provided examples. For example, two or more steps may be performed concurrently or with partial concurrence. Also, some steps that are performed as discrete steps in the following examples may be combined, and steps being performed as a combined step may be separated into discrete steps, the sequence of certain steps may be reversed or otherwise varied, and the nature or number of discrete steps may be altered or varied. Accordingly, the provided examples are not intended to exclude any of such means of obtaining a serum providing at least a portion of the proteins of the formulations.

Likewise, different reagents, techniques, materials and/or equipment other than those specifically mentioned may be utilized to provide the serum from which all or a portion of the proteins of the formulations may be isolated.

A serum providing at least a portion of the proteins of the formulations may be produced by stressing a co-culture including proliferative cells. The co-culture of cells may be obtained by first establishing a monolayer of cells on a surface. After a monolayer of a first culture is established, a second culture comprising more resilient and/or aggressive cells may be seeded on the monolayer and established. Additional cultures comprising increasingly dominant cells may then be seeded and established until a monolayer the having the desired cellular composition is obtained. The monolayer is then stressed to obtain a serum by conditioning a collection medium. All or a portion of the desired proteins of the formulations may then be isolated from the serum and combined with a suitable cosmetic base to provide a dermatological composition.

For instance, a serum comprising Human Neonatal Fibroblast/Keratinocyte Conditioned Media may be obtained from a proliferative monolayer comprising a co-culture of keratinocytes and fibroblasts. The co-cultured monolayer may be established by first partially submerging a vial of frozen keratinocytes (e.g., obtained from LifeLine Cell Technologies) in a 37° C. water bath, without submerging the top of the vial. The vial is allowed to thaw in the water bath until a small piece of ice remains. The vial is then removed and sprayed with an ethanol solution. In a hood, keratinocytes are seeded from the vial at 2,500 to 5,000 cells per cm² onto a culture treated surface. The surface should be provided with an appropriate volume of a suitable growth media, such as a media including Basal DermaLife Media (LifeLine Cell Technologies) and growth factors comprising bovine pituitary extract, L-glutamine, hydrocortisone hemisuccinate, transforming growth factor, insulin, epinephrine and/or ApoTransferrin. The seeded surface is then placed in an incubator and grown at 37° C. in the presence of humidified air comprising 5% CO₂. As to remove any residue DMSO and/or other solvents that may be present in the cryogenic solution, the growth media may be changed every 24 to 48 hours following initiation of the monoculture. After which time, the growth media may be changed every 48 to 72 hours.

Other means of obtaining the initial keratinocytes may also be employed. For instance, keratinocytes may be isolated from neonatal foreskin retrieved from circumcision using the techniques detailed in U.S. Pat. No. 9,907,745, filed Jan. 15, 2015, the contents of which are hereby incorporated by reference in their entirety.

The keratinocytes are allowed to grow in the growth media until 80-90% confluence is achieved. Voids are then created within the established monolayer by removing the growth media and washing twice with an appropriate volume of a buffer solution, such as phosphate buffer solution without calcium or magnesium. After washing with buffer solution, a sufficient volume of an enzymatic cell detachment solution to promote detachment of the cells from the surface is added. For instance, detachment of the cells may be promoted by adding a sufficient volume of a non-mammalian (non-porcine, non-bovine), non-bacterially derived dissociation solution, such as 1 ml of Accutase, available from Innovative Cell Technologies, Inc, per 25 cm² of growth area. The enzymatically treated monolayer may then be incubated at 37° C. until the keratinocytes start balling. The surface is then tilted to collect the enzyme solution with a pipette. The collected solution is sprayed at focused points onto the monolayer to create voids in about 50% of the monolayer. The enzyme solution and detached keratinocytes are then removed. The remaining monolayer is provided with a sufficient volume of the growth media and returned to the incubator. For example, an amount of media providing 10 ml of media per 55 cm² of growth area may be sufficient.

Simultaneously, a monolayer of fibroblasts is cultured on a second surface by submerging a vial of frozen fibroblasts (e.g., obtained from LifeLine Cell Technologies) in a 37° C. water bath, without submerging the top of the vial. The vial is allowed to thaw in the water bath until a small piece of ice remains. The vial is then removed and sprayed with an ethanol solution. In a hood, fibroblasts are seeded from the vial at 2,500 to 5,000 cells per cm² on to a culture treated surface. The surface should be provided with an appropriate volume of a suitable growth media, such as a media including Basal DermaLife Media (LifeLine Cell Technologies) and growth factors comprising L-glutamine, hydrocortisone hemisuccinate, lineolic acid, licithin, human serum albumin, basic fibroblasts growth factor, epidermal growth factor, transforming growth factor, insulin and/or vitamin C. The seeded surface is then placed in an incubator and grown at 37° C. in the presence of humidified air comprising 5% CO₂. As to remove any residue DMSO and/or other solvents that may be present in the cryogenic solution, the growth media may be changed every 24 to 48 hours following initiation of the monoculture. After which time, the growth media may be changed every 48 to 72 hours.

Other means of obtaining the initial fibroblasts may also be employed. For instance, fibroblasts may be isolated from neonatal foreskin retrieved from circumcision using the techniques detailed in U.S. Pat. No. 9,907,745.

When the fibroblast monolayer reaches approximately 80 to 90% confluence, the surface is transferred to a hood and the growth media removed. The fibroblasts monolayer is then washed with a buffer solution, such as phosphate buffer solution without calcium or magnesium. A sufficient volume of a cell detachment solution to promote detachment of the fibroblasts from the second surface is then added. For instance, detachment of the fibroblasts may be promoted by adding 1 ml of Accutase, manufactured by Innovative Cell Technologies, Inc, per 25 cm² of growth area. The fibroblast cells are then incubated in the cell detachment solution at 37° C. until all the cells have detached. A homogenous suspension of cells is then obtained by mixing and approximately 250 μl of the fibroblasts suspension is over-seeded onto the keratinocyte monolayer. The over-seeded keratinocyte monolayer is then returned to the incubator. The co-culture is then grown in the keratinocyte growth media until 80 to 95% confluence is achieved.

A monolayer of co-cultured cells may also be achieved by culturing keratinocytes in the keratinocyte growth media until approximately 50% confluence is achieved. The keratinocyte monolayer may then be over-seeded with the cultured fibroblasts suspension. For example, a co-culture in a T175 cm² flask would be overlayed with 1.5 ml of fibroblast suspension generated from a confluent T75 cm² flask of fibroblasts dissociated using 3 ml of Accutase. The over-seeded keratinocyte monolayer may then be cultured in the incubator until approximately 80 to 95% confluence is achieved.

The co-culture of cells may be stressed to provide a serum providing at least a portion of the proteins of the formulations. Stressing the co-culture may be achieved by selectively removing nutrients, growth factors and/or other favorable conditions. The stress need not be severe. Accordingly, sufficient stress may be induced by removing all or a portion of the growth factors while maintaining nutrient levels. Growth factors may be removed by extracting the keratinocyte growth media from the surface and rinsing the co-culture monolayer twice with a sufficient volume of a buffer solution, such as phosphate buffer solution lacking calcium and magnesium. As to ensure all growth factors are removed, the co-culture may be incubated for a period of time in a collection medium that is added to the surface and then discarded prior to serum collection. For instance, growth factors may be removed prior to serum collection by adding approximately 5.0 ml of a collection medium per 55 cm² of growth area and incubating for approximately six hours.

The collection medium may comprise a minimum essential medium with Earl's salt and have the nutrients of the keratinocyte growth medium.

After removal of the growth factors, a sufficient volume of fresh collection medium is added, and the surface returned to the incubator for a sufficient period of time to produce a conditioned medium form the collection medium. For example, incubating the co-culture in approximately 10.0 ml of fresh collection media per 55 cm² of growth area for approximately 48 hours may be sufficient to produce a conditioned media from the collection media. After incubating for a sufficient period of time, approximately 50% of the collection media is removed and replaced with an approximately equal amount of fresh collection media. The co-culture is then incubated for approximately 48 hours to produce more conditioned media. After which time, all of the conditioned media is removed.

The co-culture is then allowed to recover by removing the stress and incubating for a period of time. For instance, incubating in the presence of approximately 10.0 ml per 55 cm² of growth area of the keratinocyte growth media for approximately 24 to 72 hours may provide sufficient recovery. During recovery, the co-culture may be refreshed by seeding fresh cells of one or more of the cultures onto the monolayer.

After recovering, serum collection is repeated.

The process of the serum collection and recovery may be repeated until the co-cultures no longer produce serum of the desired quality. The quality of serum may begin to degrade when one or more of the cultures used to initially establish the co-culture reach 80% of their life expectancy as defined by the maximum number of population doublings.

The conditioned medium collected may be filtered using a suitable filter, such as a 0.45 μm Millipore filter. The serum collected from filtering the conditioned medium may be tested for sterility, virology and/or stability factors.

At least a portion of the proteins of the desired formulations may then be isolated from the serum and combined with a suitable solvent, such as water, to produce one or more of the formulations. The formulations may then be combined with a suitable cosmetic base as to provide a dermatological composition useful as a recovery cream, moisturizer, neck cream, eye cream and/or facial cream. After combining the formulations to the base, antioxidants, and/or fragrances may be added to the dermatological composition. The dermatological composition may comprise approximately 58 to 77 percent by mass of base, approximately 2 to 5 percent by mass of a moisturizer, approximately 9 to 33 percent by mass of the serum, 3 to 14 percent by mass of antioxidants, and approximately 0 to 0.01 percent by mass of fragrance. Other amounts and/or ingredients collectively providing a dermatologically suitable composition may be utilized in combination with the formulations.

In an exemplary embodiment, a facial cream dermatological composition may be prepared by adding 2.35 mass percent super low molecular weight hyaluronic acid added to 58.92 mass percent base. The base and hyaluronic acid combination may then be slowly mixed until homogenous. After which, 29.45 mass percent of one or more of the formulations may be added to the base and hyaluronic acid mixture and slowly mixed until a homogenous mixture is obtained. Then 5.89 mass percent vitamin C may be slowly mixed in to provide a homogenous mixture. After providing a homogenous mixture including vitamin C, 2.94 mass percent vitamin E may be slowly mixed in to provide a homogenous mixture. Then 0.44 mass percent vitamin A may be mixed in to provide a final homogenous vitamin-base-moisturizer-serum composition. To this composition, 0.01 mass percent fragrance may be mixed in to provide a final homogenous dermatological composition.

In an exemplary embodiment, an eye cream dermatological composition may be prepared by combining 61.83 mass percent base with 30.90 mass percent of one or more of the formulations and slowly mixing until a homogenous mixture of base and serum is obtained. To this mixture, 3.707 mass percent super low molecular weight hyaluronic acid may be slowly mixed in to provide a homogenous mixture. Then, 3.09 mass percent vitamin E may be slowly mixed in to provide another homogenous mixture. To this mixture, 0.463 mass percent vitamin A may be slowly mixed in to provide a homogenous vitamin-base-moisturizer-serum composition. To this composition, 0.01 mass percent fragrance may be slowly mixed in to provide a final homogenous dermatological composition.

In an exemplary embodiment, a neck cream dermatological composition may be prepared by combining 67.243 mass percent base with 16.810 mass percent of one or more of the formulations, and mixing slowly to provide a homogenous mixture of base and serum. To this mixture 4.203 mass percent super low molecular weight hyaluronic acid may be slowly mixed in to provide a homogenous mixture. Then, 7.985 mass percent vitamin C may be slowly mixed in to provide a homogenous mixture. To this homogenous mixture, 3.329 mass percent vitamin E may be slowly mixed in to provide another homogenous mixture. After which, 0.42 mass percent vitamin A may be slowly mixed in to provide a homogenous vitamin-base-moisturizer-serum composition. To this composition, 0.01 mass percent fragrance may be mixed in to provide a final homogenous dermatological composition.

In an exemplary embodiment, a moisturizer cream dermatological composition may be prepared by combining 76.100 mass percent base with 9.605 mass percent of one or more of the formulations and mixing slowly until a homogenous base-serum mixture is obtained. To this mixture, 3.42 mass percent super low molecular weight hyaluronic acid may be slowly mixed to provide another homogenous mixture. To this mixture, 6.60 mass percent vitamin C may be slowly mixed in to provide a homogenous mixture. After which, 3.805 mass percent vitamin E may be slowly mixed in to provide a homogenous mixture. Then, 0.46 mass percent vitamin A may be slowly mixed in to provide a homogenous vitamin-base-serum-moisturizer composition. To this composition, 0.01 mass percent fragrance may be mixed in to provide a dermatological composition.

In an exemplary embodiment, a recovery cream dermatological composition may be prepared by combining 66.67 mass percent base with 33.33 mass percent of one or more of the formulations and mixing slowly until homogenous.

The dermatological composition may comprise a therapeutic effective amount of an angio-modifying formulation comprising agrin isoform 6 and calpastatin isoform 6. The amount of agrin peptides may be less than the amount of calpastatin peptides within the formulation, with a preferred ratio of 8:13.

In addition to agrin isoform 6 and calpastatin isoform 6, the angio-modifying formulation may further comprise at least one of:
Protein Jagged-1, with the amount of protein-jagged-1 peptides being less than the amount of calpastatin peptides and less than the amount of agrin peptides, a preferred ratio of 5:13 with respect to calpastatin peptides;
Isoform 2 of Growth arrest-specific protein 6, with the amount of growth arrest-specific protein 6 peptides being less than the amount of calpastatin peptides and less than the amount of agrin peptides, a preferred ratio of 7:13 with respect to calpastatin peptides;
Vascular endothelial growth factor C, with the amount of vascular endothelial growth factor peptides being less than the amount of calpastatin peptides and more than the amount of agrin peptides, a preferred ratio of 10:13 with respect to calpastatin peptides;
72 kDa type IV collagenase, with the amount of collagenase peptides being greater than the amount of calpastatin peptides and more than the amount of agrin peptides, a preferred ratio of 22:13 with respect to calpastatin peptides; and
Desmoglein-2, with the amount of desmoglein-2 peptides being less than the amount of calpastatin peptides and less than the amount of agrin peptides, a preferred ratio of 3:13 with respect to calpastatin peptides.

The dermatological composition may comprise a therapeutic effective amount of morphogenesis formulation comprising tenascin isoform 4 and fibronectin isoform 3. The amount of tenascin peptides may be less than the amount of fibronectin peptides, with a preferred ratio of 8:41.

In addition to tenascin isoform 4 and fibronectin isoform 3, the morphogenesis formulation may further comprise at least one of:
Transforming growth factor beta induced protein ig-h3, with the amount of ig-h3 peptides being less than the amount of fibronectin peptides and more than the amount of tenascin peptides, a preferred ratio of 31:41 with respect to fibronectin peptides;
Plasminogen activator inhibitor 1, with the amount of plasminogen activator inhibitor 1 peptides being less than the amount of fibronectin peptides and more than the amount of tenascin peptides, a preferred ratio of 38:41 with respect to fibronectin peptides;
Amyloid beta A4 protein, with the amount of amyloid beta A4 peptides being than the amount of fibronectin peptides and more than the amount of tenascin peptides, a preferred ratio of 18:41 with respect to fibronectin peptides;
Glucose-6-phosphate isomerase, with the amount of isomerase peptides being less than the amount of fibronectin peptides and more than the amount of tenascin peptides, a preferred ratio of 19:41 with respect to fibronectin peptides;
Long isoform of serine protease inhibitor Kazal-type 5, with the amount of serine protease inhibitor being less than the amount of fibronectin peptides and more than the amount of tenascin peptides, a preferred ratio of 9:41 with respect to fibronectin peptides;
Cadherin-3, with the amount of cadherin-3 peptides being less than the amount of fibronectin peptides and more than the amount of tenascin peptides, a preferred ratio of 9:41 with respect to fibronectin peptides;
Pappalysin-1, with the amount of pappalysin-1 peptides being less than the amount of fibronectin peptides and less than the amount of tenascin peptides, a preferred ratio of 4:41 with respect to fibronectin peptides;
Insulin-like growth factor-binding protein 7, with the amount insulin-like growth factor-binding protein 7 peptides being less than the amount of fibronectin peptides and more than the amount of tenascin peptides, a preferred ratio of 24:41 with respect to fibronectin peptides;
Kallikrein-10, with the amount of kallikrein-10 peptides being less than the amount of fibronectin peptides and more than the amount of tenascin peptides, a preferred ratio of 22:41 with respect to fibronectin peptides;
Protocadherin fat 1, with the amount of protocadherin fat 1 peptides being less than the amount of fibronectin peptides and less than the amount of tenascin peptides, a preferred ratio of 1:41 with respect to fibronectin peptides;
Syntenin-1, with the amount of syntenin-1 peptides being less than the amount of fibronectin peptides and more than the amount of tenascin peptides, a preferred ratio of 14:41 with respect to fibronectin proteins;
Proliferation-associated protein 2G4, with the amount of proliferation-associated protein peptides being less than the amount of fibronectin peptides and more than the amount of tenascin peptides, a preferred ratio of 10:41 with respect to fibronectin peptides;
Protein CYR61, with the amount of protein CYR61 peptides being less than the amount of fibronectin peptides and more than the amount of tenascin peptides, a preferred ratio of 10:41 with respect to fibronectin peptides;
Keratinocyte proline-rich protein, with the amount of proline-rich protein peptides being less than the amount of fibronectin peptides and less than the amount of tenascin peptides, a preferred ratio of 6:41 with respect to fibronectin peptides;
Brain-specific serein protease 4, with the amount of serine protease peptides being less than the amount of fibronectin peptides, a preferred ratio of 8:41 with respect to fibronectin peptides;
Cadherin 13 isoform 4, with the amount of cadherin 13 peptides being less than the amount of fibronectin peptides and less than the amount of tenascin peptides, a preferred ratio of 4:41 with respect to fibronectin peptides;
Integrin alpha-2, with the amount of integrin alpha 2 peptides being less than the amount of fibronectin peptides and less than the amount of tenascin peptides, a preferred ratio of 2:41 with respect to fibronectin peptides;
Integrin beta-1, with the amount of beta-1 peptides being less than the amount of fibronectin peptides and less than the amount of tenascin peptides, a preferred ratio of 3:41 with respect to fibronectin peptides; and
Neural cell adhesion molecule L1 isoform 2, with the amount of adhesion molecule peptides being less than the amount of fibronectin peptides and less than the amount of tenascin peptides, a preferred ratio of 2:41 with respect to fibronectin peptides.

The dermatological composition may comprise a therapeutic effective amount of an extracellular matrix formulation comprising laminin subunit alpha 3.

In addition to laminin alpha subunit 3, the extracellular matrix formulation may further comprise at least one of:
Laminin subunit gamma 2, with the amount of gamma 2 peptides being greater than the amount of alpha 3 peptides, a preferred ratio of 42:30;
Laminin subunit beta 3, with the amount of beta 3 peptides being greater than the amount of alpha 3 peptides, a preferred ratio of 36:30;
Collagen alpha-1(I), with the amount of alpha-1(I) peptides being less than the amount of alpha 3 peptides, a preferred ratio of 11:30;
Collagen alpha-1(III), with the amount of alpha-1(III) peptides being less than the amount of alpha 3 peptides, a preferred ratio of 4:30;
Collagen alpha-1(VI), with the amount of alpha-(VI) peptides being less than the amount of alpha 3 peptides, a preferred ratio of 28:30;
Collagen alpha-2(I), with the amount of alpha-2(I) peptides being less than the amount of alpha 3 peptides, a preferred ratio of 15:30;
Collagen alpha-2(VI), with the amount of alpha-2(VI) peptides being less than the amount of alpha 3 peptides, a preferred ratio of 11:30;
Laminin subunit alpha 4, with the amount of alpha 4 peptides being less than the amount of alpha 3 peptides, a preferred ratio of 7:30;
Laminin subunit beta 1, with the amount of beta 1 peptides being less than the amount of alpha 3 peptides, a preferred ratio of 8:30;
Laminin subunit gamma 1, with the amount of gamma 1 peptides being less than the amount of alpha 3 peptides, a preferred ratio of 12:30;
Laminin 332; and
Laminin 411.

The dermatological composition may comprise a therapeutic effective amount of an immune promoting formulation comprising serapin B7, complement component C1s and complement component C3. The amount of C1s peptides may be less than the amount of complement C3 peptides and the amount of serapin B7 peptides may be more than the amount of the C3 peptides, with a preferred ratio of C1s to C3 peptides 12:14, and a preferred ratio of serapin peptides to C3 peptides of 18:14.

In addition to serapin B7, complement component C1s and complement component C3, the immune promoting formulation may further comprise at least one of:
Elafin, with the amount of elafin peptides being greater than the amount of serpin, C3 or C1s peptides, a preferred ratio of 74:14 with respect to C3 peptides;
Matrix metalloproteinase-9, with the amount of metalloproteinase peptides being less the amount of serpin, C3 or C1s peptides, a preferred ratio of 9:14 with respect to C3 peptides;
Stromelysin-2, with the amount of stromelysin-2 peptides being less the amount of serpin, C3 or C1s peptides, a preferred ratio of 9:14 with respect to C3 peptides;
HLA class I histocompatibility antigen, Cw-6 alpha chain, with the amount Cw-6 alpha chain peptides being less the amount of serpin, C3 or C1s peptides, a preferred ratio of 6:14 with respect to C3 peptides;
Quinone oxidoreductase PIG3, with the amount PIG3 peptides being less the amount of serpin, C3 or C1s peptides, a preferred ratio of 10:14 with respect to C3 peptides;
Superoxide dismutase, with the amount of dismutase peptides being greater than the amount of C1s peptides and less than the serapin peptides, a preferred ratio of 1:1 with respect to C3 peptides;
Metallothionein-2, with the amount of metallothionein peptides being more than the amount of C1s, C3 or serapin peptides, a preferred ratio of 42:14 with respect to C3 peptides;
Alpha-1 antichymotrypsin, with the amount of antichymotrypsin peptides being less the amount of serpin, C3 or C1s peptides, a preferred ratio of 6:14 with respect to C3 peptides;
Interleukin-1 receptor-like 1, with the amount of interleukin-1 receptor-like peptides being less the amount of serpin, C3 or C1s peptides, a preferred ratio of 4:14 with respect to C3 peptides; and
Neutrophil chemotactic agents.

The Neutrophil chemotactic agents may comprise at least one of:
Interleukin-6, with the amount of interleukin-6 peptides being more the amount of serpin, C3 or C1s peptides, a preferred ratio of 23:14 with respect to C3 peptides;
Growth regulated alpha protein, with the amount of alpha protein peptides being more the amount of serpin, C3 or C1s peptides, a preferred ratio of 32:14 with respect to C3 peptides;
Protein S100-A8, with the amount S100-A8 peptides being more the amount of serpin, C3 or C1s peptides, a preferred ratio of 30:14 with respect to C3 peptides;
Interleukin-8, with the amount of interleukin-8 peptides being more the amount of serpin, C3 or C1s peptides, a preferred ratio of 26:14 with respect to C3 peptides; and
C-X-C motif chemokine 5, the amount of chemokine peptides being more the amount of serpin, C3 or C1s peptides, a preferred ratio of 19:14 with respect to C3 peptides.

The dermatological composition may comprise a therapeutic effective amount of an extracellular matrix modification formulation comprising interstitial collagenase and stomelysin-1. The amount of stomelysin-1 peptides may be less than the amount of collagenase peptides, with a preferred ratio of 46:72.

In addition to interstitial collagenase and stomelysin-1, the extracellular matrix modification formulation may further comprise at least one of:
Cathepsin L2, with that amount of cathepsin peptides less than the amount of stomelysin peptides and less than the amount of collagenase peptides, a preferred ratio of 37:72 with respect to collagenase peptides;
Latent transforming growth factor beta-binding protein 2, with the amount of latent transforming growth factor beta-binding protein peptides less than the amount of stomelysin peptides and less than the amount of collagenase peptides, a preferred ratio of 6:72 with respect to collagenase peptides;
Aminopeptidase N, with the amount of aminopeptidase peptides peptides less than the amount of stomelysin peptides and less than the amount of collagenase peptides, a preferred ratio of 6:72 with respect to collagenase peptides;
Decorin, with the amount of decorin peptides less than the amount of stomelysin peptides and less than the amount of collagenase peptides, a preferred ratio of 27:72 with respect to collagenase peptides;
Urokinase-type plasminogen activator, with the amount of plasminogen activators peptides less than the amount of stomelysin peptides and less than the amount of collagenase peptides, a preferred ratio of 20:72 with respect to collagenase peptides;
Lumican, with the amount lumican peptides less than the amount of stomelysin peptides and less than the amount of collagenase peptides, a preferred ratio of 21:72 with respect to collagenase peptides;
Cystatin-M, with the amount of cystatin peptides less than the amount of stomelysin peptides and less than the amount of collagenase peptides, a preferred ratio of 30:72 with respect to collagenase peptides;
Lysyl oxidase homolog 2, with the amount of homolog peptides less than the amount of stomelysin peptides and less than the amount of collagenase peptides, a preferred ratio of 5:72 with respect to collagenase;
Cystatin-C, the amount of cystatin peptides less than the amount of stomelysin peptides and less than the amount of collagenase peptides, a preferred ratio of 25:72 with respect to collagenase peptides;
Protein-lysine 6-oxidase, the amount of oxidase peptides less than the amount of stomelysin peptides and less than the amount of collagenase peptides, a preferred ratio of 8:72 with respect to collagenase peptides;
Tissue factor pathway inhibitor 2, the amount of inhibitor peptides less than the amount of stomelysin peptides and less than the amount of collagenase peptides, a preferred ratio of 14:72 with respect to colleganase; and
Procollagen-lysine, 2-oxoglutarate 5-dioxgenase isoform 2, the amount of dioxgenase peptides less than the amount of stomelysin peptides and less than the amount of collagenase peptides, a preferred ratio of 3:72 with respect to colleganase.

The dermatological composition may comprise a cytoskeleton formulation comprising translationally-controlled tumor protein.

In addition to translationally-controlled tumor protein, the cytoskeleton formulation may further comprise at least one of:
Filamin A, with the amount of filamin peptides being less than amount translationally-controlled tumor protein peptides, a preferred ratio of 13:44;
Alpha-actin-1, with the amount of alpha-actin peptides being less than amount translationally-controlled tumor protein peptides, a preferred ratio of 31:44;
Microtubule associated protein 4, with the amount of microtubule associated protein peptides being less than amount translationally-controlled tumor protein peptides, a preferred ratio of 19:44;
Moesin, with the amount of moesin peptides being less than amount translationally-controlled tumor protein peptides, a preferred ratio of 35:44;
Vinculin, with the amount of vinculin peptides being less than amount translationally-controlled tumor protein peptides, a preferred ratio of 11:44;
Involucrin, with the amount involucrin peptides being less than amount translationally-controlled tumor protein peptides, a preferred ratio of 18:44;
Gelsolin isoform 2, with the amount gelsolin peptides being less than amount translationally-controlled tumor protein peptides, a preferred ratio of 10:44;
PDZ and LIM domain protein 1, with amount of PDZ and LIM domain protein peptides being less than amount translationally-controlled tumor protein peptides, a preferred ratio of 20:44;
Caldesmon isoform 5, with the amount of caldesmon peptides being less than amount translationally-controlled tumor protein peptides, a preferred ratio of 13:44;
LIM domain and actin-binding protein 1, with the amount of LIM domain and actin-binding protein peptides being less than amount translationally-controlled tumor protein peptides, a preferred ratio of 7:44;
Myosin regulatory light chain 12B, with the amount light chain peptides being less than amount translationally-controlled tumor protein peptides, a preferred ratio of 21:44;
Small proline rich protein 3, with the amount protein 3 peptides being less than amount translationally-controlled tumor protein peptides, a preferred ratio of 22:44;
Smooth muscle isoform of myosin light polypeptide 6, with the amount of myosin peptides being less than amount translationally-controlled tumor protein peptides, a preferred ratio of 23:44;
Jupiter microtubule associated homolog 1, with the amount of Jupiter peptides being less than amount translationally-controlled tumor protein peptides, a preferred ratio of 21:44;
Small proline rich protein 2A, with the amount of protein 2A peptides being less than amount translationally-controlled tumor protein peptides, a preferred ratio of 45:44; and
Myotrophin, with the amount of myotrophin peptides being less than amount translationally-controlled tumor protein peptides, a preferred ratio of 22:44.

The dermatological composition may further comprise at least one of:
A chaperone formulation comprising at least one of 78 kDa glucose-regulated protein and heat shock 70 kDA protein to assist protein folding and/or assembly;
Nidogen-1 to maintain the basement membrane;
Serum albumin; and
A metabolism formulation comprising at least one of pyruvate kinase, phospholipid transfer protein,

While the present invention has been described herein with respect to the exemplary embodiments, it will become apparent to one of ordinary skill in the art that many modifications, improvements and sub-combinations of the various embodiments, adaptations and variations can be made to the invention without departing from the spirit and scope thereof.

What is claimed:

1. A dermatological composition, comprising:
a therapeutic effective amount of an angio-modifying formulation comprising agrin isoform 6 and calpastatin isoform 6.

2. The composition of claim 1, wherein the angio-modifying formulation further comprises jagged-1

3. The composition of claim 1, wherein the angio-modifying formulation further comprises isoform 2 of growth arrest-specific protein 6.

4. The composition of claim 1, wherein the angio-modifying formulation further comprises vascular endothelial growth factor C.

5. The composition of claim 1, further comprising a therapeutic effective amount of a morphogenesis formulation comprising tenascin isoform 4 and fibronectin isoform 3.

6. The composition of claim 5, wherein the morphogenesis formulation further comprises transforming growth factor beta induced protein ig-h3.

7. The composition of claim 5, wherein the morphogenesis formulation further comprises a long isoform Kazal-type 5 serine protease inhibitor.

8. The composition of claim 5, wherein the morphogenesis formulation further comprises a plasminogen activator inhibitor 1 serein protease inhibitor.

9. The composition of claim 5, wherein the morphogenesis formulation further comprises receptor type tyrosine-protein phosphatase kappa.

10. The composition of claim 5, wherein the morphogenesis formulation further comprises keratinocyte proline-rich protein.

11. The composition of claim 5, wherein the morphogenesis formulation further comprises pappalysin-1.

12. The composition of claim 1, further a therapeutic effective amount of an extracellular matrix formulation comprising laminin subunit alpha 3.

13. The composition of claim 12, wherein the extracellular matrix formulation further comprises laminin subunit beta-3.

14. The composition of claim 12, wherein the extracellular matrix formulation further comprises laminin subunit gamma-2.

15. The composition of claim 12, wherein the extracellular matrix formulation further comprises laminin-332.

16. The composition of claim 1, further comprising a therapeutic effective amount of an immune promoting formulation comprising serapin B7, complement component C1s and complement component C3.

17. The composition of claim 16, wherein the immune promoting formulation further comprises a neutrophil chemotactic agent.

18. The composition of claim 1, further comprising a therapeutic effective amount of an extracellular matrix modification formulation comprising interstitial collagenase and stomelysin-1.

19. The composition of claim 18, wherein the extracellular matrix modification formulation further comprises latent-transforming growth factor beta-binding protein 2

20. The composition of claim 18, wherein the extracellular matrix modification formulation further comprises tissue factor pathway inhibitor 2.

21. A method of treating a skin condition comprising:
applying to the skin a dermatological composition comprising a therapeutic effective amount of at a least one an angio-modifying formulation, a morphogenesis formulation, an extracellular matrix formulation, an extracellular matrix modification formulation, an immune promoting formulation and a cytoskeleton formulation,
wherein the skin condition comprises at least one of wrinkles, eczema, acne, impetigo, psoriasis, rashes, rosacea and hives.

22. The method of claim 21, wherein the dermatological composition comprises a therapeutic effective amount of an angio-modifying formulation comprising agrin isoform 6 and calpastatin isoform 6.

23. The method of claim 21, wherein the dermatological composition comprises a therapeutic effective amount of a morphogenesis formulation comprising tenascin isoform 4 and fibronectin isoform 3.

24. The method of claim 21, wherein the dermatological composition comprises a therapeutic effective amount of an extracellular matrix formulation comprising laminin subunit alpha 3.

25. The method of claim 21, wherein the dermatological composition comprises a therapeutic effective amount of an immune promoting formulation comprising serapin B7, complement component C1s and complement component C3.

26. The method of claim 21, wherein the dermatological composition comprises a therapeutic effective amount of an extracellular matrix modification formulation comprising interstitial collagenase and stomelysin-1.
